(19) United States
(12) Patent Application Publication
(10) Pub. No.: US 2008/0120039 A1
(43) Pub. Date: May 22, 2008

(54) HORMONE AND DRUG CONCENTRATIONS IN THE PORTAL VEIN AND HEPATIC SINUSOIDS

(75) Inventors: Kevin Hall, Edmonton (CA); David Polidori, Rancho Santa Fe, CA (US); Scott Siler, Castro Valley, CA (US)

Correspondence Address: ENTELOS, INC.;c/o Law Offices of Karen E. Flick, P.O. Box 515, El Granada, CA, 94018-0515, US

(73) Assignee: Entelos, Inc.

(21) Appl. No.: 11/618,383

(22) Filed: Dec. 29, 2006

Related U.S. Application Data
(60) Provisional application No. 60/755,724, filed on Dec. 29, 2005.

(51) Int. Cl. G06F 19/00; A61B 5/00

(52) U.S. Cl. 702/19; 600/300

(57) ABSTRACT

The invention relates to novel methods of informing a medical decision by determining a hormone or drug concentration in a portal vein or hepatic sinusoid of a mammal. The invention also relates to methods of determining hormone or drug concentration in the portal vein or hepatic sinusoids. Further the invention relates to methods of utilizing these concentrations to calculate infusion rates for hormone or drugs in order to achieve a desired portal vein or hepatic sinusoid concentration of such a hormone or drug.

### I. INTRODUCTION

### A. CROSS-REFERENCE TO RELATED APPLICATIONS

This application claims benefit of U.S. Application Ser. No. 60/755,724, filed 29 Dec. 2005, incorporated herein by reference in its entirety.

### B. BACKGROUND OF THE INVENTION

The present invention relates generally to the field of determining hormone and drug concentrations of hormones in the portal vein and sinusoidal space of the liver.

Glucagon and insulin are secreted into the portal vein by cells in the pancreatic islets. Glucagon is secreted in response to amino acids or hypoglycemia and insulin is secreted in response to elevated glucose, amino acids, and incretin hormones. The portal vein drains into the hepatic sinusoids where these hormones have opposite effects on liver metabolism. Insulin increases glycogen synthesis and reduces hepatic glucose output whereas glucagon increases glycogenolysis, gluconeogenesis, and hepatic glucose output.

It is difficult to measure concentrations of glucagon and insulin in the portal vein and the hepatic sinusoids. It is also difficult to estimate these concentrations based on more easily sampled measurements from venous plasma because a significant amount of these hormones are removed from the sinusoidal space and degraded in the liver. Therefore, it remains challenging to predict how hepatic glucagon and insulin concentrations during hormone infusion studies compare to their values under physiological conditions.

In this invention, a new physiologically-based kinetic model for glucagon and insulin is presented that enables portal and sinusoid concentrations to be calculated based on parameters that can be more easily measured experimentally.

### II. SUMMARY OF THE INVENTION

One aspect of the invention provides methods of informing a medical decision by determining a concentration of a drug or hormone in a portal vein or hepatic sinusoid of a mammal, said method comprising a) determining a systemic plasma hormone concentration, a rate of change of systemic plasma hormone concentration as a function of exogenous hormone infusion rate, a fractional extraction by liver in steady-state conditions, and a portal vein flow rate or a hepatic vein flow rate; b) calculating the hormone concentration in the portal vein or hepatic sinusoid; and c) reporting the hormone concentration to a medical professional. Preferably the hormone is a pancreatic hormone, more preferably the hormone is insulin or glucagon.

The present invention enables portal and sinusoidal hormone, e.g., glucagon and insulin, concentrations and endogenous hormone production rates to be estimated under dynamic conditions using hormone concentrations measured from the systemic circulation. These estimates can inform a medical decision, e.g. relating to the effects of pancreatic hormones on hepatic metabolism. More particularly, determining portal vein and hepatic sinusoid concentrations of insulin and/or glucagon can inform medical decisions regarding the therapeutic approaches for type 2 diabetes and the use of therapeutic agents such as, e.g., insulin secretagogues, glucagon antagonists, exogenous insulin delivery. In certain implementations, the methods of the invention can determine the hepatic sinusoid or portal vein concentrations of inhibitors, substrates or modulators of glucokinase, glucagon receptor, glycogen phosphorylase, PDH kinase, fructose 1,6 bisphosphatase, HMG-CoA reductase, PPAR-α.

In certain implementations, the hormone or drug concentration in the portal vein (P*) is calculated using the formula:

$${P}^{*} = ( 1 + \frac{1}{m p ( 1 - f )} ) {L}^{*} - \frac{I}{p ( 1 - f )}$$

wherein m is the rate of change of systemic plasma hormone or drug concentration as a function of exogenous hormone or drug infusion rate, p is the portal vein flow rate, f is the fractional extraction by liver in steady-state conditions, L* is the systemic plasma hormone or drug concentration; and I is the exogenous hormone or drug infusion rate. The concentration of pancreatic hormones, such as insulin or glucagon, or of therapeutic agents, such as PPAR-α agonists can be determined according to the methods of the invention.

In another implementation of the invention, the hormone or drug concentration in the hepatic sinusoids is calculated using the formula:

$${S}^{*} = ( 1 + \frac{1}{mv} - f ) {L}^{*} - \frac{I}{v}$$

wherein m is the rate of change of systemic plasma hormone or drug concentration as a function of exogenous hormone or drug infusion rate, v is the hepatic vein flow rate, f is the fractional extraction by liver in steady-state conditions, L* is the systemic plasma hormone or drug concentration and I is the exogenous hormone or drug infusion rate.

Another embodiment of the invention provides methods of maintaining a basal concentration of a hormone in hepatic sinusoids in a mammal, said method comprising (a) determining a rate of change of systemic plasma hormone concentration as a function of exogenous hormone infusion rate, a fractional extraction of the hormone by liver in steady-state conditions, a hepatic vein flow rate; and fractional reduction in endogenous hormone release; (b) calculating an infusion rate of the hormone necessary to maintain the basal hormone concentration, wherein the calculation includes the rate of change, the fractional extraction, the flow rate and the fraction reduction determined in step (a); and (c) administering the hormone at the calculated rate to the mammal.

In certain implementation, the basal concentration corresponds to a fasted state (L_{fasting}). In which case, the target systemic plasma concentration is calculated as:

$${L}_{soma}^{*} = {L}_{fasting}^{*} ( 1 + \frac{δ}{mv ( 1 - f )} )$$

wherein m is the rate of change of systemic plasma hormone concentration as a function of exogenous hormone infusion rate, v is the hepatic vein flow rate, f is the fractional extraction by liver in steady-state conditions, δ is the fractional reduction in endogenous hormone release and L* is the systemic plasma hormone concentration. The infusion rate can be calculated using Equations 4 or 5, described herein. Preferably, the fractional reduction in endogenous hormone release is due to somatostatin infusion.

Yet another aspect of the invention provides methods of determining a concentration of a hormone or drug in a portal vein or hepatic sinusoid of a mammal, said method comprising a) determining a systemic plasma hormone concentration, a rate of change of systemic plasma hormone concentration as a function of exogenous hormone infusion rate, a fractional extraction by liver in steady-state conditions, and a portal vein flow rate or a hepatic vein flow rate; and b) calculating the hormone concentration in the portal vein or hepatic sinusoid.

In certain implementations of the invention, the hormone or drug concentration in the portal vein (P*) is calculated using the formula:

$${P}^{*} = ( 1 + \frac{1}{m p ( 1 - f )} ) {L}^{*} - \frac{I}{p ( 1 - f )}$$

wherein m is the rate of change of systemic plasma hormone or drug concentration as a function of exogenous hormone or drug infusion rate, p is the portal vein flow rate, f is the fractional extraction by liver in steady-state conditions, L* is the systemic plasma hormone or drug concentration, and I is the exogenous hormone or drug infusion rate.

In an alternative implementation of the invention, the hormone concentration in the hepatic sinusoids (S*) is calculated using the formula:

$${S}^{*} = ( 1 + \frac{1}{mv} - f ) {L}^{*} - \frac{I}{v}$$

wherein m is the rate of change of systemic plasma hormone or drug concentration as a function of exogenous hormone or drug infusion rate, v is the hepatic vein flow rate, f is the fractional extraction by liver in steady-state conditions, L* is the systemic plasma hormone or drug concentration, and I is the exogenous hormone or drug infusion rate.

It will be appreciated by one of skill in the art that the embodiments disclosed herein may be used together in any suitable combination to generate additional embodiments not expressly recited above, and that such embodiments are considered to be part of the present invention

### III. BRIEF DESCRIPTION OF THE DRAWINGS

FIG. 1 provides a graphical representation of an exemplary kinetic model of hormone dynamics, in this case, of glucagon. The physical variables are shown in the top figure and the corresponding mathematical symbols are shown in the bottom figure.

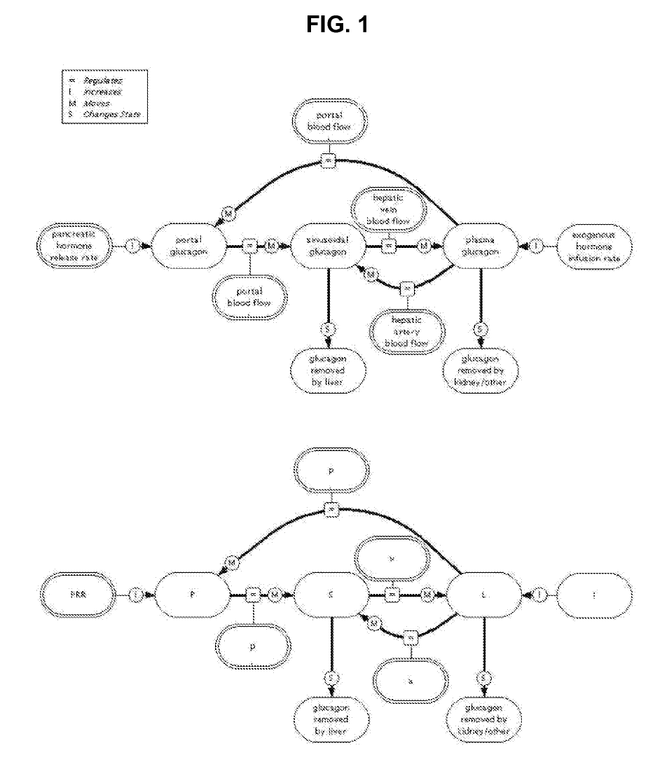

FIG. 2 provides a graph of systemic plasma insulin concentrations developed in response to insulin infusions with somatostatin. Data points are from the scientific literature (Basu, et al., Diabetes 39(2):272-283 (2000), Clore, et al., Am. J. Physiol. Encroinol. Metab. 287(2):E358-65 (August 2004, e-published Apr. 13, 2004), Vella, et al., Diabetologia 45(10):1410-1415 (2002) and Adkins, et al., Diabetes 52(9):2213-2220 (2003)) and assume a 70 kg individual with 20% body fat. The equation for the regression line is y=0.7x with r²=0.98.

FIGS. 3A and 3B provide graphs of plasma glucagon concentrations during different glucagon infusion rates in human subjects. FIG. 3A illustrates data from studies that report lower glucagon concentrations during glucagon infusions (Chhibber, et al. Metabolism 49(1):39-46 (2000) and Matsuda, et al. Metabolism 51(9):1111-1119 (2002)) and FIG. 3B illustrates data from studies that report higher glucagon concentrations during glucagon infusions (Nielsen, et al., Diabetes 46(12):2007-2016 (1997) and Andrews, et al., J. Clin. Endocrinol. Metab. 88(1):285-291 (2003)). The equation for the regression line in FIG. 3A is y=0.41x+60.15 with r²=0.96 and the regression line in FIG. 3B is y=x+69.5 with r=0.97.

FIGS. 4A to 4D provide graphs of specified endogenous glucagon release and portal blood flow to simulate a mixed meal (A and B) and an oral glucose load (C and D) given at time t=60 minutes.

FIGS. 5A-5F provide graphs of plasma, portal, and sinusoidal glucagon concentrations determined by the kinetic model in response to a simulated mixed meal (A, B, C) and an oral glucose load (D, E, F) as specified in FIG. 4. In each simulation, the meal is specified at time t=60 minutes.

FIG. 6 provides a graph of portal and plasma glucagon concentrations following a mixed meal at t=0 in one of the subjects studied by Dencker et al., Scan. J. Gastroenterol. 10(5):471-474 (1975). Note that portal glucagon levels decrease after meal consumption despite a 40-50% increase in plasma levels. The plasma concentration was not reported at t=30.

### IV. DETAILED DESCRIPTION

Several compartmental models for insulin and glucagon kinetics have previously been reported (Alford, et al. J. Clin. Endocrinol. Metab. 42(5):830-838 (1976), Dobbins, et al. Metabolism 44(4):452-459 (1995), and Cobelli, et al., Diabetes 37(2):223-231 (1988)). These models have generally been used to determine the metabolic clearance rates of the hormones and the number of compartments, distribution volumes, and transfer rates that are most consistent with results from studies measuring hormone clearance following infusions. In contrast, the model developed here provides estimates of portal and sinusoidal hormone concentrations based on measurements taken from the systemic circulation.

The invention provides methods of determining a hormone or drug concentration in a portal vein or hepatic sinusoid of a mammal, said method comprising a) determining a systemic plasma hormone or drug concentration, a rate of change of systemic plasma hormone or drug concentration as a function of exogenous hormone or drug infusion rate, a fractional extraction by liver in steady-state conditions, and a portal vein flow rate or a hepatic vein flow rate; and b) calculating the hormone or drug concentration in the portal vein or hepatic sinusoid.

The methods of the invention are based on a model describing the kinetics of secretion, distribution, and clearance of pancreatic hormones. The model is physiologically-based and makes use of reported hepatic blood flow rates to determine the rates of transfer of hormones from the portal vein to the liver and into the circulation. This model contains a minimal number of assumptions and most parameters in the model have been reported in the scientific literature (see, e.g., Szinnai, et al., Scand. J. Gastroenterol. 36(5):540-545 (2001), Dauzat, et al., Eur. J. Appl. Physiol. Occup. Physiol. 68(5):373-380 (1994), and Olefsky, et al. Eur. J. Clin. Invest. 4:1217 (1974)). Algebraic relationships were derived to show that the unknown parameters could be determined based on values of two more easily measured parameters, the fractional hormone or drug extraction by the liver and the metabolic clearance rate (or its reciprocal, m).The model comprises three compartments for each hormone or drug: portal vein, hepatic sinusoids, and plasma in the systemic circulation (the “plasma compartment”). The following assumptions included in the model of the invention as follows:
1. Each compartment is well-mixed.
2. The rate of transfer of a hormone or drug from one compartment A into another compartment B is equal to the product of the rate of blood flow between the compartments and the concentration of the hormone or drug in compartment A.
3. Hormones or drugs are removed from the sinusoid compartment (by liver uptake and degradation) and from the plasma compartment (by clearance from other tissues, including the kidney). The clearance rates are proportional to the concentration of the hormones or drugs in these compartments. There is no clearance in the portal vein.

In addition, the model utilizes the average insulin and glucagon secretion rates and does not take into account the pulsatility of hormone release.

The three differential equations describing the kinetics of each hormone or drug are

$$\begin{matrix}{V}_{P} \frac{d P}{d t} = PRR + pL - pP & ( 1 ) \\ {V}_{S} \frac{d S}{d t} = pP + aL - vS - \frac{{V}_{s}}{{τ}_{s}} S & ( 2 ) \\ {V}_{L} \frac{d L}{d t} = I + vS - ( p + a ) L - \frac{{V}_{L}}{{τ}_{L}} L & ( 3 )\end{matrix}$$

P=portal hormone or drug concentration
S=sinusoidal hormone or drug concentration
L=systemic plasma hormone or drug concentration
p=portal blood flow rate (ml/min)
a=hepatic artery blood flow rate (ml/min)
v=hepatic vein blood flow rate (ml/min)
V_{P}=portal volume (ml)
V_{S}=sinusoidal volume (ml)
V_{L}=systemic plasma volume (ml)
PRR=pancreatic hormone release rate
I=exogenous hormone or drug infusion rate
τ_{S}=time constant for clearance from liver (min)
τ_{L}=time constant for clearance from plasma (min)
t=time
   P, S, and L have units of μU/ml for insulin and pg/ml for glucagon; PRR and I have units of μU/min for insulin and pg/min for glucagon. The time constants are equal to the half-life in the compartment divided by ln 2.

Under steady-state conditions (e.g., overnight fasted individuals, sufficiently long constant hormone infusions), all of the derivatives (and hence the left-hand sides) in Equations 1-3 are zero. In this case, we can solve for the steady-state concentrations of hormone or drug in each compartment, which will be denoted P*, S*, and L*.

Equations 1-3 contain several parameters that have been reported frequently (p, a, v, V_{P}, V_{S}, V_{L}) and several others with values that are not as well-known (τ_{S}, τ_{L}, PRR). In addition, concentrations in the systemic plasma, L*, are easily measured, but the values in the portal vein, P*, and in the sinusoidal space, S, are not easily measured and hence are not as well characterized.

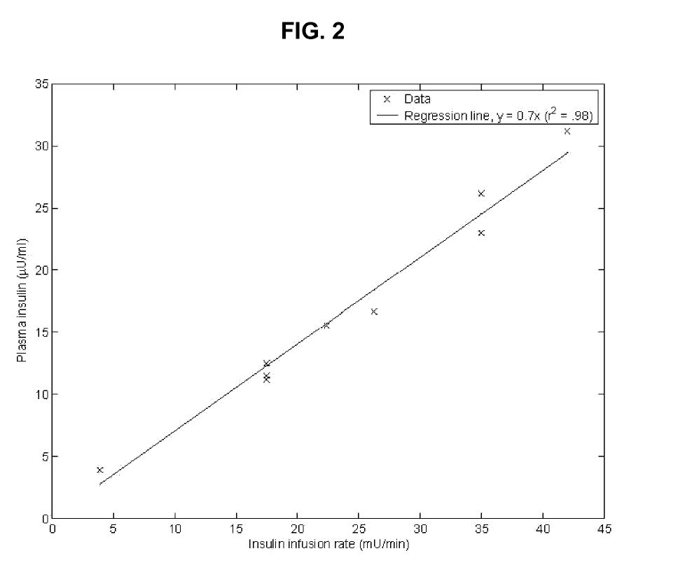

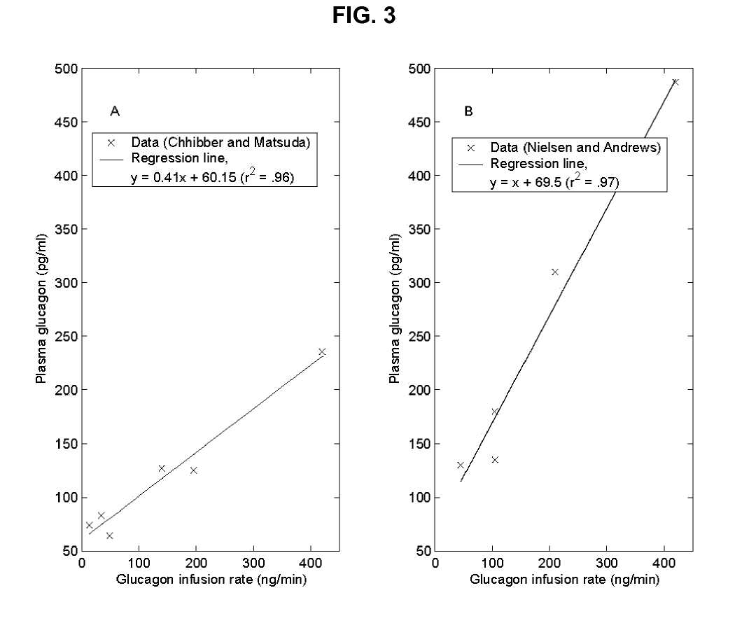

Data from hormone infusion studies can be used to determine the unknown parameters in Equations 1-3. This can be done most effectively in situations where endogenous hormone release is well-controlled, which is generally accomplished experimentally by intravenous infusion of somatostatin. Data from several studies illustrating the administration of multiple insulin infusions in the same patients with combination with somatostatin are provided in FIG. 2. Similarly, administration of multiple glucagon infusions in the same patients in combination with somatostatin are illustrated in FIG. 3. For the glucagon infusions, considerably different results are obtained from different studies; these results can be collected into two groups, a lower glucagon concentration group and a higher glucagon concentration group, as illustrated in FIGS. 3A and 3B, respectively.

In each of the studies in FIGS. 2 and 3, patients were infused with two or more different rates of insulin or glucagon combined with an unchanging infusion of somatostatin. Because the same somatostatin infusion was used in each patient, it can be assumed that the endogenous insulin and glucagon release rates were similar in each study. Thus, as described below in Example 1, the following equations relating portal and sinusoidal hormone concentrations to their systemic plasma concentrations can be derived.

$${P}^{*} = ( 1 + \frac{1}{m p ( 1 - f )} ) {L}^{*} - \frac{I}{p ( 1 - f )} \qquad ( 4 )$$

$${S}^{*} = ( 1 + \frac{1}{mv} - f ) {L}^{*} - \frac{I}{v} \qquad ( 5 )$$

where m is the slope of the line relating steady-state plasma concentrations to exogenous infusion rates (which can be read from the graphs in FIGS. 2 and 3) and f is the fractional extraction of the hormone by the liver. m can also be described as the rate of change of systemic plasma hormone concentration as a function of exogenous hormone infusion rate.

By definition, m is also the reciprocal of the metabolic clearance rate (MCR) for the hormone. Once m has been determined, the other unknown quantities in Equations 1-3 can be determined from the following equations (see Example 1 for derivation)

$$\begin{matrix}{τ}_{L} = \frac{{mV}_{L}}{1 - mvf} & ( 6 ) \\ {τ}_{S} = \frac{{V}_{s} ( 1 - f )}{vf} & ( 7 ) \\ {PRR}_{fasting} = \frac{{L}_{fasting}^{*}}{m ( 1 - f )} & ( 8 )\end{matrix}$$

where PRR_{fasting} and L* fasting are the endogenous hormone release rate and systemic plasma concentration, respectively, in the overnight fasted condition. Additionally, the ratios of portal and sinusoidal hormone concentrations to systemic plasma concentrations in the overnight fasted state can be determined by setting I=0 in Equations 4 and 5, giving

$${( \frac{{P}^{*}}{{L}^{*}} )}_{fasting} = 1 + \frac{1}{m p ( 1 - f )} \qquad ( 9 )$$

$${( \frac{{S}^{*}}{{L}^{*}} )}_{fasting} = 1 + \frac{1}{mv} - f \qquad ( 10 )$$

For insulin, the reported value of m in healthy individuals is approximately 0.0007 min/ml. Using this value, equations derived from the model provide predictions for portal insulin concentrations and endogenous insulin release consistent with reported values. This is not the case for glucagon, however. Results from different published studies in healthy individuals give considerably different values for m. The studies can be grouped into sets, one in which m≈0.0004 min/ml and another in which m≈0.001 min/ml. Interestingly, the group of studies that give the lower value of m infused somatostatin at approximately twice the rate as in the studies giving the higher value of m. This observation suggests that somatostatin may alter glucagon clearance. Variability in the subject population is probably not the major cause of the observed differences because results from Matsuda et al. (Metabolism 51(9):1111-1119 (2003)) yield nearly identical values of m for healthy patients and patients with type 2 diabetes, and Alford et al. (J. Clin. Endocrinol. Metab. 42(5):830-838 (1976)) reported similar metabolic clearance rates for glucagon in healthy and diabetic subjects. The choice of m=0.001 is within the range of reported metabolic clearance rates of glucagon and leads to a predicted ratio of portal to systemic plasma glucagon concentration that is also within the range of what has been reported (see, e.g., Jaspan, et al., J. Clin. Endocrinol. Metab. 58(2):287-292 (1984), Blackard, et al., Diabetes 23(3):199-202 (1974), Hayakawa, et al., Surg. Today 28(4):3630366 (1998) or Dencker, et al., Scand. J. Gastroenterol. 10(5):471-474 (1975)).

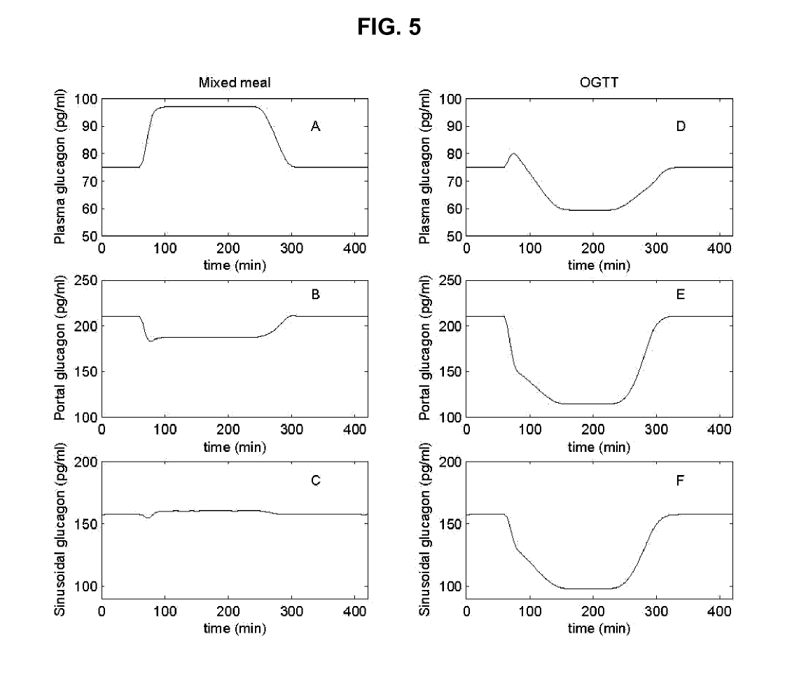

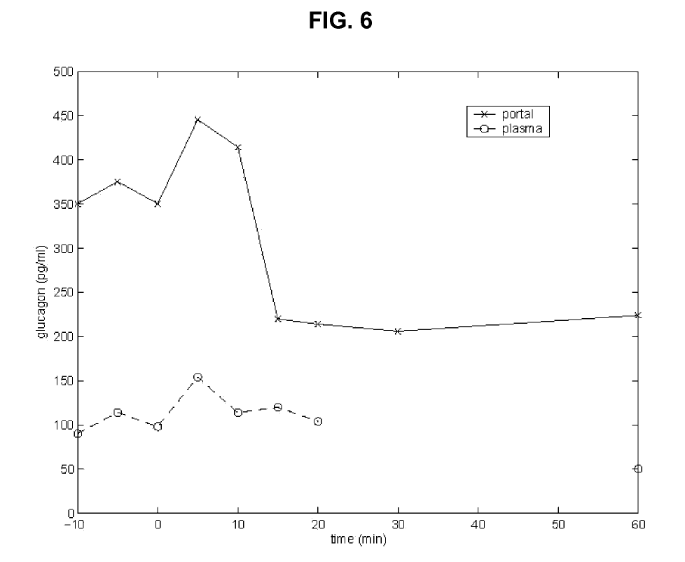

In addition to determining portal and sinusoidal hormone concentrations in the overnight fasted state, the new kinetic model can be used to determine portal and sinusoidal hormone concentrations in the postprandial state. The predictions for glucagon concentrations following a mixed meal or an oral glucose load (FIG. 5) demonstrate that changes in portal and sinusoidal concentrations can be considerably different than changes in plasma concentrations. Interestingly, portal concentrations were predicted to decrease even though plasma concentrations increased following the mixed meal. Postprandial portal glucagon concentrations in humans have only been reported in one study with three subjects. Although there was considerable variability in the response of each of the three patients to the meal, one of the patients responded very similarly to predictions from the model in both overnight and postprandial conditions as shown in FIG. 6. The difference between the postprandial portal and plasma glucagon response in this patient was not described by the authors.

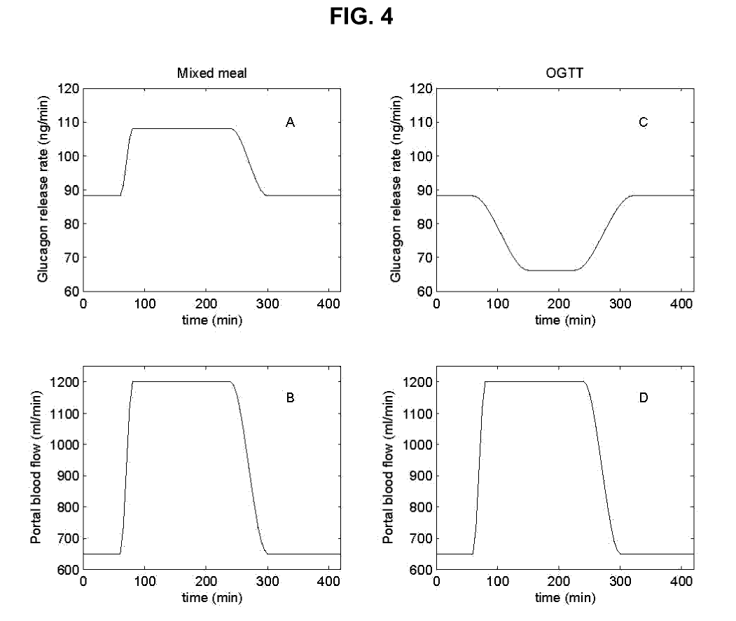

The methods of the invention can be used to provide novel calculations for postprandial hormone concentrations in the portal vein and hepatic sinusoids. Following a meal, portal blood flow typically increases approximately two-fold. Plasma glucagon concentrations in healthy subjects have been observed to increase by about 30% following a mixed meal and to decrease by about 20% following an oral glucose load. In order to determine portal glucagon concentrations during these conditions, portal blood flow was specified to increase two-fold in the postprandial state and the endogenous release rate was specified to give the desired plasma glucagon profile in the postprandial state. The profiles specified for portal blood flow and endogenous glucagon release are shown in FIG. 4. The other parameters for the kinetic model were the same as in Case 2 above, where m=0.001 min/ml.

Results under these conditions are shown in FIG. 5. Plasma glucagon concentrations achieve the desired profiles, with plasma glucagon increasing approximately 30% following the mixed meal and decreasing approximately 20% following the oral glucose load. An unanticipated prediction is that portal glucagon concentration decreases following a mixed meal and sinusoidal glucagon concentrations remain roughly constant, even though systemic plasma concentrations increase by approximately 30%. Additionally, it is predicted that the portal glucagon concentrations decrease by approximately 50% and the sinusoidal concentrations decrease by approximately 40% following oral glucose even though systemic plasma concentrations decrease by only 20%.

The methods of the invention can also be applied to water soluble drugs that are absorbed into the portal vein to estimate concentrations of the drugs in the portal vein and hepatic sinusoids based on measurements of the drug concentration in the systemic circulation.

The invention also provides methods of determining the appropriate infusion rate and plasma hormone concentration necessary to obtain a desired hormone concentration in the portal vein or hepatic sinusoids. For example, in studies investigating hormonal regulation of hepatic metabolism, it is often desired to maintain a basal concentration of at least one of the hormones in the hepatic sinusoids. Because somatostatin decreases endogenous insulin and glucagon release, an exogenous infusion of hormone is required to restore the hormone to basal levels. By combining Equations A7 and A19 in Example 1, it can be shown that sinusoidal hormone concentrations will be restored to overnight fasted values during a somatostatin infusion if

$${L}_{soma}^{*} = {L}_{fasting}^{*} ( 1 + \frac{δ}{mv ( 1 - f )} ) \qquad ( 11 )$$

where L*ₛₒₘₐ is the systemic plasma concentration during the somatostatin infusion and δ is the fractional reduction in endogenous hormone release due to somatostatin. Somatostatin infusions typically inhibit endogenous insulin release nearly completely and inhibit glucagon release by about 70%, giving δ=1 for insulin and δ=0.3 for glucagon. Applying Equations 5 and 11 to glucagon with m=0.001, f=0.15, and L*_{fasting}=75 pg/ml suggests that in order to restore sinusoidal glucagon concentrations to basal levels during somatostatin infusions, glucagon should be infused at a rate of I≈55 ng/min, giving a systemic concentrations of L*ₛₒₘₐ≈108 pg/ml.

Various medical infusion pumps are commercially available. Insulin pumps, for example, are widely used by diabetics. An insulin pump is a device that periodically dispenses very small amounts of insulin (or suitable insulin analogs) according to a preprogrammed profile set by the user to cover basal insulin needs. Basal insulin stimulates disposal of glucose produced by the body on a continuous basis. When a diabetic person consumes food, the diabetic person needs to estimate the amount of insulin required to cover the carbohydrates, and perhaps other food components such as protein, and program the pump to administer a bolus amount of insulin sufficient to cover the food. Typically, many insulin pump users compute the amount of carbohydrates in the food, and, using an individual carbohydrate/insulin ratio, calculate the magnitude of the bolus. However, the methods of the present invention can be used to direct a medical infusion pump to provide an amount of hormone or drug required to obtain desired drug or hormone concentrations in the portal and systemic pools. In a preferred embodiment, the determination of the amount of hormone or drug to be infused can be based upon a combination of standard values, e.g., δ=1 for insulin or portal vein blood flow being 650 ml/min, and measured values, e.g. current hormone or drug plasma levels.

Thus, the invention also provides a medical infusion pump for delivering hormone or drug doses to a subject, said pump comprising an actuator in the medical infusion pump coupled to the infusion pump processor suitable for delivering medicament doses to a user; a data receiver suitable for measuring plasma hormone or drug concentrations or for receiving a plasma hormone concentration from user input or an automated device; a medicament storage; and a processor, wherein the processor determines a concentration of the hormone or drug in portal vein or hepatic sinusoid based on the plasma hormone or drug concentration received by the data receiver and further wherein the processor calculates the amount of hormone or drug required to obtain a desired drug or hormone concentration in the portal vein or hepatic sinusoid.

### V. EXAMPLES

### A. Example 1

### Equation Derivations

In steady-state conditions, equations (1-3) become

0=PRR+pL*−pP* (A1)

$$0 = {pP}^{*} + {aL}^{*} - {vS}^{*} - \frac{{V}_{S}}{{τ}_{S}} {S}^{*} \qquad ( A2 )$$

$$0 = I + {vS}^{*} - {vL}^{*} - \frac{{V}_{L}}{{τ}_{L}} {L}^{*} \qquad ( A3 )$$

where P*, S, and L* are the equilibrium concentrations of glucagon in the portal vein, sinusoids, and systemic plasma. Equation A3 has been simplified by using the relationship p+a=v (portal blood flow+hepatic artery blood flow=hepatic vein blood flow).

Equation A1 can be arranged to give

$${P}^{*} = {L}^{*} + \frac{PRR}{p} \qquad ( A4 )$$

The equations can be simplified by defining f to be the fractional extraction by the liver in steady-state conditions, which has been measured experimentally for insulin and glucagon.

$$\begin{matrix}f = \frac{clearance by liver}{input to liver} \\ = \frac{clearance by liver}{clearance by liver + unextracted hormone leaving liver}\end{matrix}$$

### Therefore,

$$f = \frac{\frac{{V}_{S}}{{τ}_{S}} {S}^{*}}{\frac{{V}_{S}}{{τ}_{S}} {S}^{*} + {vS}^{*}} = \frac{\frac{{V}_{S}}{{τ}_{S}}}{\frac{{V}_{S}}{{τ}_{S}} + v} \qquad ( A5 )$$

### Rearranging gives

$$\frac{{V}_{S}}{{τ}_{S}} = v \frac{f}{1 - f} ⇒ v + \frac{{V}_{S}}{{τ}_{S}} = v + v \frac{f}{1 - f}$$

which simplifies to

$$v + \frac{{V}_{S}}{{τ}_{S}} = \frac{v}{1 - f} \qquad ( A6 )$$

### Substituting A4 into A2 gives

$${S}^{*} = \frac{{pP}^{*} + {aL}^{*}}{v + \frac{{V}_{S}}{{τ}_{S}}} = \frac{{pL}^{*} + {aL}^{*} + PRR}{v + \frac{{V}_{S}}{{τ}_{S}}}$$

### Using p+a=v and substituting Equation A6 in the above equation gives

$${S}^{*} = ( 1 - f ) {L}^{*} + \frac{1 - f}{v} PRR \qquad ( A7 )$$

### Substituting A7 into A3 gives

$$( v + \frac{{V}_{L}}{{τ}_{L}} ) {L}^{*} = I + {vS}^{*} = I + v ( 1 - f ) {L}^{*} + ( 1 - f ) PRR$$

which can be simplified to

$$( vf + \frac{{V}_{L}}{{τ}_{L}} ) {L}^{*} = I + PRR ( 1 - f ) \qquad ( A8 )$$

### Substituting Equation A6 into Equation A2 yields

$${pP}^{*} = ( v + \frac{{V}_{S}}{{τ}_{S}} ) {S}^{*} - {aL}^{*} = \frac{{vS}^{*}}{1 - f} - {aL}^{*} \qquad ( A9 )$$

and equation A3 gives

$${vS}^{*} = ( v + \frac{{V}_{L}}{{τ}_{L}} ) {L}^{*} - I = ( vf + \frac{{V}_{L}}{{τ}_{L}} + v ( 1 - f ) ) {L}^{*} - I \qquad ( A10 )$$

### Adding and subtracting vf to the term in parentheses on the right hand side gives

$${vS}^{*} = ( vf + \frac{{V}_{L}}{{τ}_{L}} + v ( 1 - f ) ) {L}^{*} - I \qquad ( A11 )$$

### Substituting equation A11 into equation A9 gives

$${pP}^{*} = ( ( v - a ) + \frac{vf + \frac{{V}_{L}}{{τ}_{L}}}{1 - f} ) {L}^{*} - \frac{I}{I - f}$$

### Using v−a=p and simplifying gives

$${P}^{*} = ( 1 + \frac{1}{p ( 1 - f )} ( vf + \frac{{V}_{L}}{{τ}_{L}} ) ) {L}^{*} - \frac{I}{p ( 1 - f )} \qquad ( A12 )$$

Equation A12 relates portal and plasma hormone concentrations but still contains the unknown parameter τ_{L}. The next section describes a method for determining a related parameter from experimental studies.

### B. Example 2

### Multiple Infusions with Somatostatin

In several experimental situations, multiple infusions of the hormones are given to individuals who are also receiving somatostatin infusions. To consider how the steady state systemic plasma concentration, L*, changes with the infusion rate, I, Equation A8 can be rewritten as

$${L}^{*} = ( \frac{1}{vf + \frac{{V}_{L}}{{τ}_{L}}} ) ( I + PRR ( 1 - f ) )$$

### Taking the derivative of the above equation with respect to I gives

$$\frac{d {L}^{*}}{d I} = \frac{1}{vf + \frac{{V}_{L}}{{τ}_{L}}} \qquad ( A13 )$$

where it is assumed that because somatostatin was given, the endogenous hormone release rate did not change with different hormone infusion rates (i.e.

$$\frac{d PRR}{d I} = 0 ) .$$

The linearity of the data points in FIGS. 2 and 3 suggests that this assumption is valid. The value of dL*/dI can easily be obtained from experimental data. This value is simply the slope of the line obtained by plotting hormone concentration on the y-axis and infusion rate on the x-axis (see FIGS. 2 and 3 for examples of these plots). Because this value can be obtained as the slope of a line, we define

$$m = \frac{d {L}^{*}}{d I} = \frac{1}{vf + \frac{{V}_{L}}{{τ}_{L}}} \qquad ( A14 )$$

### With the definition of m, equation A12 simplifies to

$${P}^{*} = ( 1 + \frac{1}{m p ( 1 - f )} ) {L}^{*} - \frac{I}{p ( 1 - f )} \qquad ( A15 )$$

Equation A15 provides a relationship between portal and plasma concentrations in terms of the parameters m, p, and f that have been determined experimentally. Using m as defined in Equation A14 to simplify Equation A11 yields the following equation for sinusoidal concentrations

$${S}^{*} = ( 1 + \frac{1}{mv} - f ) {L}^{*} - \frac{I}{v} \qquad ( A16 )$$

Once m and f are determined, the unknown parameters τ_{S} and τ_{L} can be determined. From equation A14,

$${τ}_{L} = \frac{m {V}_{L}}{1 - mvf} \qquad ( A17 )$$

and from equation A5

$${τ}_{S} = \frac{{V}_{S} ( 1 - f )}{vf} \qquad ( A18 )$$

### In addition, the endogenous hormone release in the overnight fasted condition (PRR_{fasting}) can be determined from equation A8 with I=0 as

$${PRR}_{fasting} = \frac{{L}_{fasting}^{*}}{m ( 1 - f )} \qquad ( A19 )$$

where L*_{fasting} is the concentration of the hormone in the overnight fasted state.

### C. Example 3

### Determining Unknown Parameters for Insulin

From the studies shown in FIG. 2, m=0.0007 min/ml. This value is consistent with a number of studies that have reported metabolic clearance rates of insulin of 13-27 ml/kg/min in healthy patients; taking the reciprocal of the MCR reported in these studies gives m=0.0005-0.001 min/ml for a 70 kg patient. Reported values for the fractional extraction of insulin by the liver vary, although most estimates give f=0.5-0.7. Values of f≈0.5 have been reported from a portal catheterization study (Nygre, et al. Metabolism 34(1):48-52 (1985)) and from mathematical models based on insulin and c-peptide concentrations (Polonsky, et al., J. Clin. Invest. 82(2):435-441 (1988)), whereas splanchnic balance studies report f=0.7 (Shah, et al., Diabetes 51(2):301-310 (2002)). Solving Equations 6-8 taking m=0.0007, f=0.5, p (portal blood flow) as 650 ml/min, a (hepatic artery blood flow) as 150 ml/min, v (hepatic vein blood flow) as 800 ml/min, V_{S} (sinusoidal compartment volume, estimated to be 50% of liver volume) as 700 ml, V_{P} (volume of the portal vein) as 8 ml, and V_{L} (volume of systemic plasma) as 4200 ml, gives the following parameter values:

$${τ}_{L} = 4.1 min$$

$${τ}_{S} = 0.875 min$$

$${PRR}_{fasting} = 14.2 mU \text{/} min$$

Using these parameter values, Equations 9 and 10 predict that in the overnight fasted condition, portal insulin is approximately 5 times higher than systemic insulin and sinusoidal insulin is approximately 2.2 times higher. Thus, for an individual with a fasting insulin concentration of 5 μU/ml in the systemic plasma, the model predicts that the fasting portal insulin concentration is approximately 27 μU/ml and endogenous insulin release rate is approximately 14 mU/min. Both of these are consistent with values reported in the literature.

### D. Example 4

### Determining Unknown Parameters for Glucagon

Different research groups report considerably different glucagon concentrations in response to plasma glucagon infusions. In FIG. 3A, the regression line yields m=0.00041 min/ml, whereas for the regression line for 3B yields m=0.001 min/ml. These differences lead to considerable differences in predicted portal glucagon concentrations in both fasted and fed conditions, as described below. The MCR for glucagon has been reported to be between 7-14 ml/kg/min; taking the reciprocal of the MCR for a 70 kg individual gives m=0.001-0.002 min/ml. As a result of the variability in the reported measurements for this parameter, two different possibilities are considered in the analyses: one where m=0.00041 and another where m=0.001 min/ml.

The fractional extraction of glucagon by the liver is considerably smaller than the fractional extraction of insulin, although human data are very limited. Felig et al. (Proc. Soc. Exp. Biol. Med. 147(1):88-90 (1974)) report that there is little or no hepatic glucagon extraction in humans, although portal glucagon concentrations have been reported to be 1.3-3.0 times higher than concentrations in the systemic plasma. A portal to plasma ratio greater than 1 suggests that there is some extraction of glucagon by the liver. Reported values for hepatic glucagon extraction in animals include f≈0.1 in pigs, f≈0.2 in dogs, f≈0.07 in sheep, and f≈0.5 in rats. In the calculations below, a value of f=0.15 is used. Analysis of Equation 9 shows that predictions for the ratio of portal to systemic plasma glucagon concentrations are not very sensitive to the value of f; for example, if f varies from 0 to 0.25, the predicted ratio of portal to systemic plasma glucagon concentrations changes approximately 20%.

The ratios of portal and sinusoidal glucagon concentrations to systemic plasma concentrations in the overnight fasted state can be computed from Equations 9 and 10. Using m=0.00041 and f=0.15 yields a portal-to-plasma ratio of 5.4, which is considerably higher than the ratios of 1.3-3.0 reported in the literature and a sinusoidal-to-plasma ratio of 3.9. The other parameters in this case are τ_{L}=1.8 min, τ_{S}=5 min, and PRR_{fasting}=215 ng/min.

Repeating the analysis using m=0.001 and f=0.15 yields a portal-to-plasma ratio of 2.8, which is at the high end of the range reported in the literature. In this case, the sinusoidal-to-plasma ratio is 2.1 and the other parameters are τ_{L}=4.8 min, τ_{S}=5 min, and PRRfasting=88.2 ng/min.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the subject invention.

What is claimed is:

1. A method of informing a medical decision by determining a concentration of a hormone or drug in a portal vein or hepatic sinusoid of a mammal, said method comprising:
a) determining
(i) a systemic plasma concentration of the drug or hormone;
(ii) a rate of change of systemic plasma concentration as a function of exogenous infusion rate; and
(iii) a fractional extraction by liver in steady-state conditions;
(iv) a portal vein flow rate or a hepatic vein flow rate;
b) calculating the concentration in the portal vein or hepatic sinusoid; and
c) reporting the concentration to a medical professional.

2. The method of claim 1, wherein the hormone is a pancreatic hormone.

3. The method of claim 2, wherein the pancreatic hormone is insulin or glucagon.

4. The method of claim 1, wherein the concentration in the portal vein (P*) is calculated using the formula:
$${P}^{*} = ( 1 + \frac{1}{m p ( 1 - f )} ) {L}^{*} - \frac{I}{p ( 1 - f )}$$
wherein m is the rate of change of systemic plasma concentration as a function of exogenous infusion rate, p is the portal vein flow rate, f is the fractional extraction by liver in steady-state conditions, L* is the systemic plasma concentration, and I is the exogenous infusion rate or the drug or hormone.

5. The method of claim 1, wherein the concentration in the hepatic sinusoids (S*) is calculated using the formula:
$${S}^{*} = ( 1 + \frac{1}{mv} - f ) {L}^{*} - \frac{I}{v} .$$
wherein m is the rate of change of systemic plasma concentration as a function of exogenous infusion rate, v is the hepatic vein flow rate, f is the fractional extraction by liver in steady-state conditions, L* is the systemic plasma concentration, and I is the exogenous infusion rate of the drug or hormone.

6. A method of maintaining a basal concentration of a hormone or drug in hepatic sinusoids in a mammal, said method comprising:
(a) determining
(i) a rate of change of systemic plasma concentration as a function of exogenous infusion rate of the drug or hormone; and
(ii) a fractional extraction of the hormone by liver in steady-state conditions;
(iii) a hepatic vein flow rate; and
(iv) fractional reduction in endogenous release of the drug or hormone;
(b) calculating an infusion rate of the drug or hormone necessary to maintain the basal concentration, wherein the calculation includes the rate of change, the fractional extraction, the flow rate and the fraction reduction determined in step (a); and
(d) administering the drug or hormone at the calculated rate to the mammal.

7. The method of claim 6, wherein the hormone is a pancreatic hormone

8. The method of claim 7, wherein the pancreatic hormone is insulin or somatostatin.

9. The method of claim 6, wherein the basal concentration corresponds to a fasted state.

10. The method of claim 6, wherein a systemic plasma hormone concentration (L*ₛₒₘₐ) is calculated as
$${L}_{soma}^{*} = {L}_{fasting}^{*} ( 1 + \frac{δ}{mv ( 1 - f )} )$$
wherein m is the rate of change of systemic plasma hormone concentration as a function of exogenous hormone infusion rate, v is the hepatic vein flow rate, f is the fractional extraction by liver in steady-state conditions, δ is the fractional reduction in endogenous hormone release and L*_{fasting} is the systemic plasma hormone concentration after an overnight fast and wherein the infusion rate is calculated by solving Equation 4 or 5
$$\begin{matrix}{P}^{*} = ( 1 + \frac{1}{m p ( 1 - f )} ) {L}^{*} - \frac{I}{p ( 1 - f )} & ( 4 ) \\ {S}^{*} = ( 1 + \frac{1}{mv} - f ) {L}^{*} - \frac{I}{v} . & ( 5 )\end{matrix}$$

11. The method of claim 6, wherein the fractional reduction in endogenous hormone release is due to somatostatin infusion.

12. A method of determining a concentration or a drug or hormone in a portal vein or hepatic sinusoid of a mammal, said method comprising:
a) determining
(i) a systemic plasma hormone concentration;
(ii) a rate of change of systemic plasma hormone concentration as a function of exogenous hormone infusion rate; and
(iii) a fractional extraction by liver in steady-state conditions;
(iv) a portal vein flow rate or a hepatic vein flow rate; and
b) calculating the hormone concentration in the portal vein or hepatic sinusoid.

13. The method of claim 12, wherein the concentration in the portal vein (P*) is calculated using the formula:
$${P}^{*} = ( 1 + \frac{1}{m p ( 1 - f )} ) {L}^{*} - \frac{I}{p ( 1 - f )}$$
wherein m is the rate of change of systemic plasma concentration as a function of exogenous infusion rate, p is the portal vein flow rate, f is the fractional extraction by liver in steady-state conditions, L* is the systemic plasma concentration; and I is the exogenous infusion rate.

14. The method of claim 12, wherein the concentration in the hepatic sinusoids (S*) is calculated using the formula:
$${S}^{*} = ( 1 + \frac{1}{mv} - f ) {L}^{*} - \frac{I}{v}$$
wherein m is the rate of change of systemic plasma concentration as a function of exogenous infusion rate, v is the hepatic vein flow rate, f is the fractional extraction by liver in steady-state conditions, L* is the systemic plasma concentration, and I is the exogenous infusion rate.
